(19) United States
(12) Patent Application Publication
(10) Pub. No.: US 2005/0245796 A1
(43) Pub. Date: Nov. 3, 2005

(54) Non-invasive measurement of blood glucose using retinal imaging

(75) Inventors: Joe W. Woods, Lexington, KY (US); John L. Smith, Fair Play, CA (US); Mark J. Rice, Jacksonville, FL (US); Wilson Routt, Lexington, KY (US); Robert G. Messerschmidt, Corrales, NM (US); Junli Ou, Lexington, KY (US)

Correspondence Address: WILSON SONSINI GOODRICH & ROSATI, 650 PAGE MILL ROAD, PALO ALTO, CA, 94304-1050, US

(73) Assignee: Fovioptics, Inc., Santa Clara, CA (US)

(21) Appl. No.: 11/177,015

(22) Filed: Jul. 7, 2005

Related U.S. Application Data
(63) Continuation of application No. 10/863,619, filed on Jun. 8, 2004.
(60) Provisional application No. 60/477,245, filed on Jun. 10, 2003.

(51) Int. Cl. A61B 5/00

(52) U.S. Cl. 600/315

(57) ABSTRACT

An apparatus carries out measurements of blood glucose in a repeatable, non-invasive manner by measurement of the rate of regeneration of retinal visual pigments, such as cone visual pigments. The rate of regeneration of visual pigments is dependent upon the blood glucose concentration, and by measuring the visual pigment regeneration rate, blood glucose concentration can be accurately determined. This apparatus exposes the retina to light of selected wavelengths in selected distributions and subsequently analyzes the reflection (as color or darkness) from a selected portion of the exposed region of the retina, preferably from the fovea.

### CROSS-REFERENCE

This application is a continuation of Ser. No. 10/863,619, filed Jun. 8, 2004, which claims the benefit of U.S. Provisional Application No. 60/477,245 filed Jun. 10, 2003, which is incorporated herein by reference in its entirety.

### FIELD OF THE INVENTION

This invention pertains to the field of non-invasive in vivo measurement of blood analytes.

### BACKGROUND OF THE INVENTION

The measurement of blood glucose by diabetic patients has traditionally required the drawing of a blood sample for in vitro analysis. The blood sampling is usually done by the patient himself as a finger puncture, or in the case of a young child, by an adult. The need to draw blood for analysis is undesirable for a number of reasons, including discomfort to the patient, the high cost of glucose testing supplies, and the risk of infection with repeated skin punctures which results in many patients not testing their blood as frequently as recommended.

Many of the estimated three million Type I diabetics in the United States are asked to test their blood glucose up to six times or more per day in order to adjust their insulin doses for tighter control of their blood glucose levels. As a result of the discomfort, many of these patients do not test as often as is recommended by their physician, with the consequence of poor blood glucose control. This poor control has been shown to result in increased complications from this disease. Among these complications are blindness, heart disease, kidney disease, ischemic limb disease, and stroke. In addition, there is recent evidence that Type II diabetics (numbering over 10 million in the United States) may reduce the incidence of diabetes-related complications by more tightly controlling their blood glucose. Accordingly, these patients may be asked to test their blood glucose nearly as often as the Type I diabetic patients.

It would thus be desirable to obtain fast and reliable measurements of blood glucose concentration through simple, non-invasive testing. Prior efforts to obtain non-invasive blood glucose measurements have typically involved the passage of light waves through solid tissues such as the fingertip, forearm and the ear lobe and subsequent measurement of the absorption spectra. These efforts have been largely unsuccessful primarily due to the variability of absorption and scatter of the light waves in the tissues. These approaches, which have generally attempted to measure glucose concentration by detecting extremely small optical signals corresponding to the absorbance spectrum of glucose in the infrared or near-infrared portion of the electromagnetic spectrum, have suffered from the size requirements of instrumentation necessary to separate the wavelengths of light for this spectral analysis. Some groups, as illustrated by U.S. Pat. No. 6,280,381, have reported the use of diffractive optical systems, while others, as illustrated by U.S. Pat. No. 6,278,889, have used Fourier-transform or interferometric instruments. Regardless of approach, the physical size and weight of the instruments described have made it impractical for such a device to be hand-held or worn on the body as a pair of glasses. Other groups have attempted non-invasive blood glucose measurement in body fluids such as the anterior chamber of the eye, tears, and saliva. More recent developments have involved the analysis of light reflected from the retina of the eye to determine concentrations of blood analytes. See U.S. Pat. Nos. 6,305,804; 6,477,394; and 6,650,915, the disclosures of which are incorporated herein by reference.

### SUMMARY OF THE INVENTION

The present invention carries out measurements of blood glucose in a repeatable, non-invasive manner by measurement of the rate of consumption of glucose, or the rate of production of another substance which is dependent on the glucose concentration of the individual, as an indication of the individual's glucose concentration. The rate of consumption of glucose (or the rate of production of a second glucose concentration-dependent substance) can be the result of the consumption of glucose by a specific organ or part of the body, or by a specific biochemical process in the body. One such process is the rate of regeneration of retinal visual pigments, such as cone visual pigments. The rate of regeneration of visual pigments is dependent upon the blood glucose concentration, by virtue of the glucose concentration limiting the rate of production of a cofactor, NADPH, which is utilized in the rate-determining step of the regeneration of visual pigments. Thus, by measuring the visual pigment regeneration rate, blood glucose can be accurately determined. One preferred embodiment of this invention exposes the retina to light of selected wavelengths at selected times and analyzes the reflection (as color or darkness) from a selected portion of the exposed region of the retina, preferably from the fovea. In addition, since the rate of glucose consumption, or of the production of a glucose-concentration dependent substance can be indicative of illnesses, pathologies or other clinically-significant conditions of the health of the individual, embodiments of this invention can be used to screen for or to diagnose those conditions.

The light source in accordance with an embodiment of the invention that is used to generate the illuminating light is directed onto the retina by having the subject look forward (for example, at a marker) that brings the fovea into the central area of illumination and subsequent analysis. This naturally provides for the incident light striking the area of the retina where the cones (with their particular visual pigment) are located. Alternatively, the non-foveal retina may be used to measure pigment regeneration. In one embodiment of the invention, a photodetector array such as a CCD (or similar photodetector array) is used to form an image of the retina, and the light in the image from the region of the fovea is preferably used to determine the rate of regeneration of retinal pigments such as the cone visual pigments. In other embodiments of the invention, imaging is not necessary and light reflection from the region of interest on the retina can be used to calculate the regeneration rate of the visual pigments. In these embodiments, a photodetector such as a photodiode (for example) could be used in place of an array.

With either imaging or non-imaging embodiments of this invention, light may be used that varies in a selected temporal manner, such as a periodically applied stimulus of light that may break down (deplete or “bleach”) the visual pigment, and then reflected light from the retina is analyzed over a period of time to determine the regeneration rate of the visual pigment. As the pigment is depleted during bleaching, the color or darkness of the retina decreases (that is, the retina becomes lighter in color), with the result that more light is reflected by the bleached retina (resulting in increased reflectance). During regeneration, the pigment is restored, making the retina progressively darker and less reflective of light, leading to decreases in reflectance as the regeneration proceeds. Measurement of an unknown blood glucose concentration is accomplished by development of a relationship between the reflected light data (indicating the visual pigment regeneration rate) and corresponding clinically determined blood glucose concentration values. With either the imaging or non-imaging embodiments of this invention, a steady-state illuminating light or a varying illuminating light may be applied to induce bleaching and a steady-state illuminating light or a varying illuminating light may be applied to determine the regeneration rate of the visual pigment. Measurement of regeneration rate may also be accomplished during the bleaching phase, as regeneration of the visual pigments occurs continuously. In addition, measurement of visual pigment regeneration may be made without a formal bleaching event. The device can be preferably used by the patient in a self-testing mode, or the device may be used by an operator. Light modulated in a number of ways, such as by sinusoidal, square—wave or pulsed techniques, may be used to observe a number of phenomena described in the detailed description of the invention.

In accordance with the descriptions of the invention, a hand-held, stationary, or preferably a head-fitted instrument that measures the resulting data in the reflected light from a series of applied light stimuli or a steady-state light stimulus, may be utilized for the determination of the visual pigment regeneration rate and the subsequent calculation of blood glucose values.

Further objects, features, and advantages of the invention will be apparent from the following detailed description when taken in conjunction with the accompanying drawings.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a general diagram of an exemplary embodiment of a system for non-invasive measurement of blood glucose using retinal visual pigment.

FIG. 2 is a schematic diagram of an apparatus for measurement of blood glucose in accordance with an exemplary embodiment.

FIG. 3a is a representation of a pair of goggles, illustrating a potential form factor of an exemplary embodiment.

FIG. 3b is a representation of a hand-held monocular device, illustrating a potential form factor of an exemplary embodiment.

FIG. 3c is a representation of a hand-held binocular device, illustrating a potential form factor of an exemplary embodiment.

FIG. 3d is a representation of a head-mounted device, illustrating a potential form factor of an exemplary embodiment.

FIG. 4 is a schematic diagram of a further apparatus in accordance with an exemplary embodiment that incorporates a communications link to a remote processing system.

FIG. 5 is a diagram illustrating the effect of applying pulses of illuminating light to cause bleaching of visual pigments followed by pulses of lower intensity light to allow imaging and determination of the rate of regeneration of the visual pigments.

FIG. 6 is a schematic diagram of a further optical illumination and detection system that may be utilized in the apparatus of FIGS. 1 and 2.

FIG. 7 is a schematic diagram of an optical illumination and detection system that may be utilized in the apparatus of FIGS. 1 and 2.

FIG. 8 is a graph of an example reflectance trace.

FIG. 9 is an expanded view of a portion of the graph of FIG. 8, showing a trace where the subject has a relatively high glucose level.

FIG. 10 is a closer view of a portion of a reflectance trace graph where a subject has a low glucose level.

FIG. 11 is a depiction of two graphs having a linear portion of regeneration data near the beginning of a post-bleach phase, the top graph from a patient with a low glucose and the bottom graph from a patient with a high glucose.

FIG. 12 is a depiction of a sinusoidally-varying light signal used in the apparatus of FIG. 7.

FIG. 13 is a depiction of a DC component of reflectance and a sinusoidally-varying component of reflectance used in the apparatus of FIG. 7.

FIG. 14 is a depiction of AC component of reflected light and a difference signal used in the apparatus of FIG. 7.

FIG. 15 is a depiction of light pulses having increasing amplitude used in the apparatus of FIG. 7.

FIG. 16 is a depiction of constant amplitude pulses used in the apparatus of FIG. 7.

FIG. 17 is a depiction of two-frequency modulation used in the apparatus of FIG. 7.

FIG. 18 is a depiction of the “steady-state” method of glucose measurement used in the apparatus of FIG. 7.

FIG. 19 is a graph of glucose readings using the apparatus of FIG. 7 compared to glucose readings using a finger stick blood glucose measurement.

FIG. 20 is a Clarke Error Grid with measured and referenced glucose measurements using the apparatus of FIG. 7.

### DETAILED DESCRIPTION OF THE INVENTION

Rhodopsin is the visual pigment contained in the rods (that allow for dim vision) and cone visual pigment is contained in the cones of the retina (that allow for central and color vision). The outer segments of the rods and cones contain large amounts of visual pigment, stacked in layers lying perpendicular to the light incoming through the pupil. As visual pigment absorbs light, it breaks down (bleaches) into intermediate molecular forms and initiates a signal that proceeds down a tract of nerve tissue to the brain, allowing for the sensation of sight. During normal vision this bleaching process occurs continuously. Light that reacts with the visual pigments causes a breakdown of those pigments. This phenomenon is termed bleaching, since the retinal tissue loses its color content when a light is directed onto it. In addition, regeneration of the visual pigments occurs at all times, even during the bleaching process. Rod visual pigment absorbs light energy in a broad band centered at 500 nm, whereas the three different cone visual pigments or opsins have broad overlapping absorption bands peaking at 430, 550, and 585 nm, which correspond to blue, green, and red cones, respectively.

The rods and cones of the retina are arranged in specific locations in the back of the eye. The cones, which provide central and color vision, are located with their greatest density in the area of the fovea centralis in the retina. The fovea covers a circular area with a diameter of about 1.5 mm. The rods are found predominately in the more peripheral portions of the retina and contribute to vision in dim light.

Visual pigment consists of 11-cis-retinal and a carrier protein, which is tightly bound in either the outer segment of the cones or rods. 11-cis-retinal is the photoreactive portion of visual pigment, which is converted to all-trans-retinal when a photon of light in the active absorption band strikes the molecule. This process goes through a sequence of chemical reactions (called visual pigment regeneration), including all-trans-retinal isomerizing back to 11-cis-retinal. During the initial portion of this series of chemical steps, the nerve fiber, which is attached to that particular rod or cone, undergoes a stimulus that is perceived in the brain as a visual signal. During this process, an electrical signal is generated that can be measured on an electroretinogram (ERG) or electroencephalogram (EEG).

Following the conversion of 11-cis-retinal to all-trans-retinal, the 11-cis-retinal is regenerated by a series of steps that result in 11-cis-retinal being recombined with an opsin protein in the cell or disk membrane. A critical (and rate-limiting) step in this regeneration pathway is the reduction of all-trans-retinal to all-trans-retinol using the enzyme all-trans-retinol dehydrogenase (ATRD), which requires NADPH as the direct reduction energy source. In a series of experiments, Futterman et al. have proven that glucose, via the pentose phosphate shunt (PPS), provides virtually all of the energy required to generate the NADPH needed for this critical reaction. S. Futterman, et al., “Metabolism of Glucose and Reduction of Retinaldehyde Retinal Receptors,” J. Neurochemistry, 1970, 17, pp. 149-156. Without glucose or its immediate metabolites, only very small amounts of NADPH are formed and visual pigment cannot regenerate.

In addition, Ostroy, et al. have proven that the extracellular glucose concentration has a major effect on visual pigment regeneration. S. E. Ostroy, et al., “Extracellular Glucose Dependence of Rhodopsin Regeneration in the Excised Mouse Eye,” Exp. Eye Research, 1992, 55, pp. 419-423. Since glucose is the primary energy source for visual pigment regeneration, embodiments of the present invention utilize this relationship to measure blood glucose concentrations.

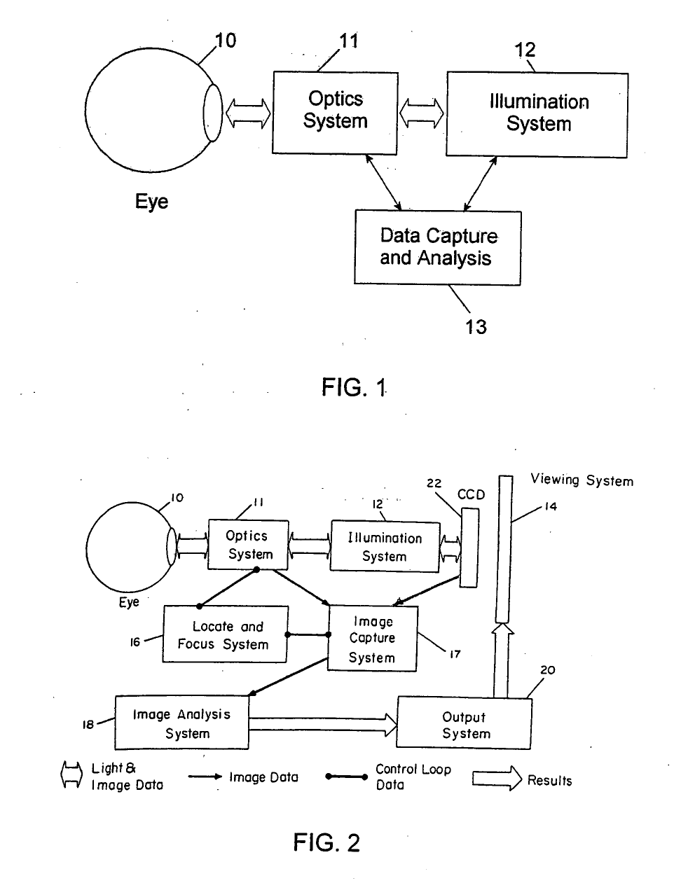

With reference to the drawings, FIG. 1 illustrates a generic embodiment of the present invention. The eye of the patient is illustrated at 10, with the optical system for directing light into the eye and obtaining light emitted from the eye shown as 11. The illumination system is shown as 12 and contains the elements required for directing light through the pupil and onto the retina for the breakdown of visual pigment regeneration (bleaching). The data capture and analysis system 13 comprises elements required for the measurement of the reflected light, calculation of the visual pigment regeneration rate, and conversion of this information into the blood glucose value.

A number of specific methodologies are described herein to make an accurate measurement of the visual pigment regeneration rate, and more than one method may be chosen depending on the particular cost and performance sought for each application.

With either imaging or non-imaging embodiments of this invention, light may be used to break down (or bleach) the visual pigment, and reflected light from the retina can be subsequently analyzed over a period of time to determine the regeneration rate of the visual pigment. Measurement of an unknown blood glucose concentration is accomplished by development of a relationship between the reflected light data (indicating the visual pigment regeneration rate) and corresponding clinically determined blood glucose concentration values. With either imaging or non-imaging embodiments of this invention, a steady-state illuminating light or a varying illuminating light may be applied to induce bleaching and a steady-state illuminating light or a varying illuminating light may be applied to determine the regeneration rate of the visual pigment. Measurement of regeneration rate may also be accomplished during the bleaching phase, as regeneration of the visual pigments occurs even while the pigments are being bleached. In addition, measurement of visual pigment regeneration may be made without a formal bleaching event. The device can be preferably used by the patient in a self-testing mode, or the device may be used by an operator. Pulsed or other light-varying techniques may be used to measure the regeneration rate of the visual pigment.

FIG. 2 illustrates an embodiment of the present invention using imaging. In this embodiment, the illumination system 12 provides selected illuminating light imaging the retina. The illumination system 12 is preferably a monochromatic or multiple discrete wavelength light source that provides light for imaging the retina. Preferably, the system provides light for imaging coaxially to reduce the likelihood of extraneous reflections from the interior or exterior of the eye. The light from the illumination system is projected through the pupil, using optics system 11. The wavelength of this light source is selected dependent upon the particular visual pigment to be analyzed. Although any visual wavelength of light could be used, the light intended for absorption by visual cone pigments could be centered at 540 nm for green cones and 585 nm for red cones. Illumination light may be composed of two (or more) separate lighting systems, such as a xenon strobe, multiple laser diodes, or light-emitting diodes (LEDs).

If the device is used with an operator, infrared imaging, which may be utilized to align the retina prior to imaging in the visual wavelengths, may be done utilizing a filtered halogen or laser diode source. The light is reflected from the retina of the eye 10 and passed through the pupil opening of the eye to the optics system 11 and through the illumination system 12 entering, e.g., a charge coupled device (CCD) or complementary metal-oxide semiconductor (CMOS) image detector 22. The illumination system 12 and optics system 11 may be similar to systems used in existing non-mydriatic fundus cameras.

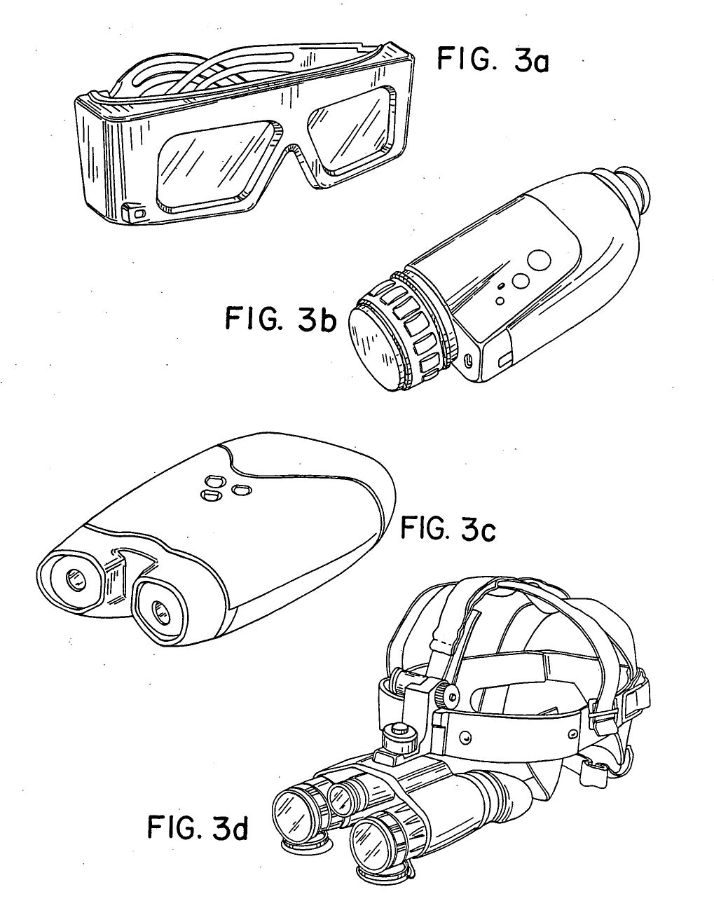

In an alternative embodiment where an operator is required, viewing system 14, for example, a liquid crystal display (LCD) screen, may receive the image data and display the image for use by the operator for initially locating the patient's retina, based on an image from the optical system in real time. A coaxial “scene” or visual target may be included in the visual field of the device so that a patient can fixate his or her eye on this scene and reduce eye motion. In addition to reducing eye motion, the location of this visual target can bring the fovea centralis into the approximate center of the CCD detector 22. In devices intended for children, the scene may include a visually pleasant object such as a familiar animal. The fixating light may also exist as a separate optical system for use with the other eye. In the currently commercially available Nidek NM100 Hand-Held Non-Mydriatic Fundus Camera, the liquid crystal display (LCD) (or other display) screen is typically located on a desktop power source that is attached to the hand-held camera by a cable. While such displays may be used in the exemplary embodiments, the LCD screen (or other display device) may be placed on the back of the hand-held camera unit, so that the operator can more easily locate the retina, having the patient's eye and the LCD screen in the same line-of-sight. The illumination system 12 and detection system 22 may include the Nidek NM100 Hand-Held Non-Mydriatic Fundus Camera, the Topcon TRC-50 EX (TRC-NW5S/TRC-NW 5SF) and Topcon TRC NW6S Non-Mydriatic Retinal Cameras, including one or two Pulnix TM-7EX CCD digital cameras to capture images at one or two wavelengths. Preferably, the device may be operated by the patient as a self-testing device. The patient may place his or her eye near the lens of the device, aligning the eye with a pre-determined spot of light or a small scene. This device may be similar in size and form to currently-marketed virtual reality or night-vision goggles, as shown in FIG. 3a. Although exemplary embodiments may be used with a dilated eye pupil, it is preferable that the imaging of the retina be carried out without requiring dilation of the pupil for speed of measurement and patient convenience. The camera may include a shield (not shown) to prevent ambient light from entering the optical system 11 to minimize extraneous reflections and the introduction of optical noise.

Referring again to FIG. 2., the optical system 11 also interfaces with a locate and focus system 16, which utilizes feedback from an image capture system 17, also interfaced to the optic system 11, to automatically find and bring the retina into focus. A convolver or other pattern recognition software may be utilized to locate the fovea. After using the pattern recognition information to more precisely locate the fovea in the center of the viewing field, the image may then be magnified using a series of lenses in the optics system 11 such that the fovea fills a large portion of the active area of the CCD (or other detector). The optical system preferably tracks the movement of the retina such that the fovea is centered and occupies most of the optical field of view. The optical system 11 may be configured to track the motion of the retina through a motor drive system that slightly gimbals the lens system. This motion system is driven and controlled in a closed loop manner utilizing the feedback of the pattern recognition software. Alternatively, if the patient is able to keep his or her eye still during the measurement, the registration of images would not be required. To adjust for variations in the individual patient's refraction, a refractive adjustment such as a variable corrective lens with a thumbwheel adjuster may be incorporated into the device. Should changes in the patient's focus change during the measurement (e.g., during naturally-occurring accommodation), the image processing or optics can be adapted to compensate. This can be done by comparing the focus of successive images, and correcting the optical system using an electromechanical servo system to adjust focal position of the optics, or by known image-processing techniques in the computing system.

The image capture system 17 is selectively controlled by the software (or alternatively by the operator) and uses feature and pattern recognition to drive the locate and auto focus system 16 to capture and store an appropriate image for analysis. Image capture itself is analogous to the function provided by a “digital still camera.” The initial image capture may be carried out with commercially available data capture boards such as a National Instruments NI 1409 installed in a computer such as a commercial PC. The image capture system 17 may utilize feature and pattern recognition to drive the locate and focus system to capture and store an appropriate image for analysis. Commercially available pattern recognition software including the mathematical tools in MATLAB may be used. An image analysis system 18 is interfaced with the image capture system 17 to analyze the light reflected from the retina to quantitatively determine the amount of glucose present. The results may be displayed to the operator via the output system 20. The output system 20 presents results together with any feedback associated with the acquisition of the data, and may include an LCD display screen or other display devices.

FIG. 3a illustrates one form factor of an analysis apparatus in conjunction with the eye of the patient, shown illustratively at 10 in FIG. 2. The analysis apparatus includes an optics system 11 comprised of lenses for projecting illuminating light onto the retina, directly through the pupil, and for receiving the light reflected from the retina passed out through the pupil, and for focusing that light to create a signal or to form an image. The glasses preferably include lensing to provide an optimal view of the retina to be illuminated and imaged. In such a system, glucose concentration information may be displayed to the user directly while the glasses are worm. When used in this form factor, in order for the device to be used conveniently by a patient, it is especially desirable that the weight and volume of the device be minimized, preferably to a weight of about ten ounces or less, and to a total volume of about twenty cubic inches or less.

FIG. 3b illustrates another form factor of an analysis apparatus in conjunction with the eye of the patient, shown illustratively at 10 in FIG. 2. The analysis apparatus includes an optics system 11 comprised of lenses for projecting illuminating light onto the retina, directly through the pupil, and for receiving the light reflected from the retina passed out through the pupil, and for focusing that light to create a signal or to form an image. The monocular device preferably includes lensing to provide an optimal view of the retina to be illuminated and imaged. In such a system, glucose concentration information may be displayed to the user directly while the monocular device is in use.

FIG. 3c illustrates another form factor of an analysis apparatus in conjunction with the eye of the patient, shown illustratively at 10 in FIG. 2. The analysis apparatus includes an optics system 11 comprised of lenses for projecting illuminating light onto the retina, directly through the pupil, and for receiving the light reflected from the retina passed out through the pupil, and for focusing that light to create a signal or to form an image. The binocular device preferably includes lensing to provide an optimal view of the retina to be illuminated and imaged. In such a system, glucose concentration information may be displayed to the user directly while the binocular device is in use.

FIG. 3d illustrates another form factor of an analysis apparatus in conjunction with the eye of the patient, shown illustratively at 10 in FIG. 2. The analysis apparatus includes an optics system 11 comprised of lenses for projecting illuminating light onto the retina, directly through the pupil, and for receiving the light reflected from the retina passed out through the pupil, and for focusing that light to create a signal or to form an image. The head-mounted device preferably includes lensing to provide an optimal view of the retina to be illuminated and imaged. In such a system, glucose concentration information may be displayed to the user directly while the head-mounted device is in use.

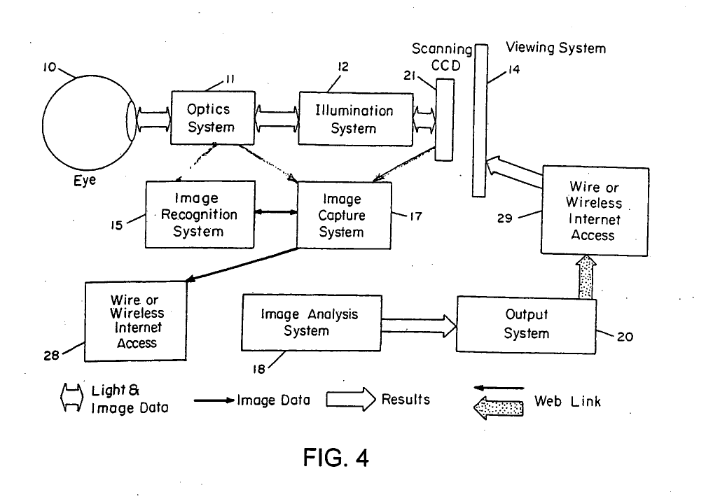

As illustrated in FIG. 4, image processing and analysis may take place at a location remote from the clinical setting by using a wired or wireless internet link (or dedicated communication link) to transfer data from the image capture system 17 to a central computer at a remote location (i.e., anywhere in the world linked by the internet) at which the image analysis system 18 is implemented. The output data from the output system 20 may be transferred back through an access link 29 to the viewing system 14 at measurement apparatus, or remote clinic (or to another location, as desired).

Following bleaching of the visual pigment with light at selected wavelengths, one embodiment uses the measurement of reflected light from the area of interest, which preferably is the fovea of the retina (although any area of the retina that contains visual pigment could be used) to measure visual pigment regeneration. The retina, at specific wavelengths of light, is illuminated as described above, and the reflected light is captured by a sensing device as described above. This sensing device may be a CCD, a CMOS imager, a photodiode or any other device that can sense the amount of light being emitted from the eye in order to measure the regeneration of the visual pigment during or following bleaching. In one embodiment using imaging, the light values of the pixels (in the case of a CCD or CMOS imager) that are in a defined area containing the desired visual pigment to be measured can then be summed. Although the exemplary embodiments can be used to measure the changing light reflected off any defined area in the retina of the eye, it is preferred to measure the foveal area which contains the highest percentage of cones compared to rods. Although both cones and rods contain visual pigment, the regeneration of cone pigment is considered to be faster than rod visual pigment regeneration and therefore preferable for measurement of regeneration rates. The highest concentration of cone visual pigment is contained in the area of the fovea, which is the area of central vision. Since several exemplary embodiments of this invention measure regeneration of visual pigment, the reflected light must be measured over a period of time, either with constant light or via a series of pulses. One embodiment makes the measurement of visual pigment regeneration with a series of pulses. This temporal measurement can be accomplished by comparing the reflected illumination from pulse to pulse, over a series of pulses, of the same area of the retina. A better estimate of the changing reflectance may be made by averaging the change in reflectance over a number of pulses to minimize noise. Although a large number of pulses may be used for greatest accuracy, it is generally desirable to use as few pulses as possible for patient convenience and comfort. A pulse is defined as any illumination of the retina, which may be a temporal illumination with any intensity, modulation and frequency. In addition, the illumination may be a steady-state illumination.

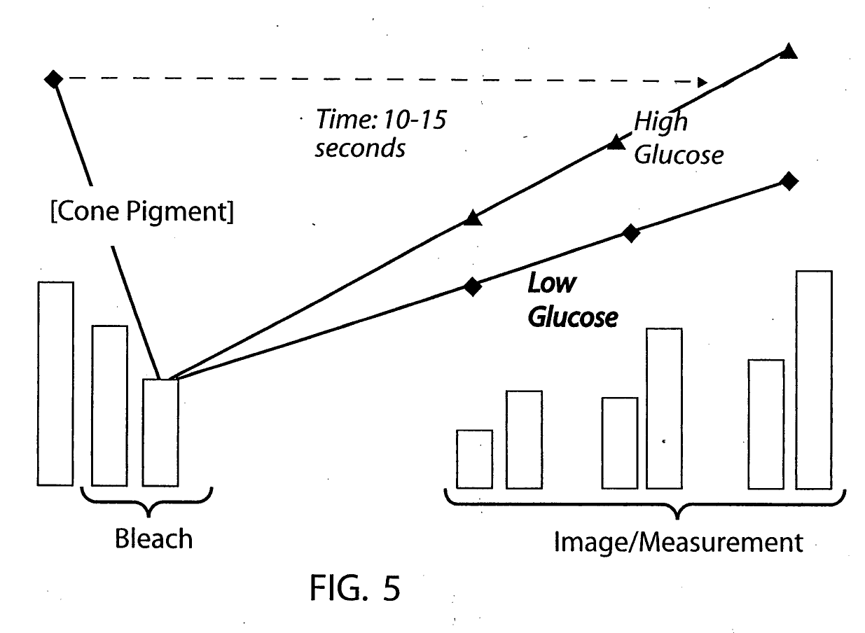

Various pulse sequences may be utilized comprising, for example, a pulse or series of pulses at wavelengths of light that cause the breakdown (bleaching) of the visual pigment, and then a series of pulses (possibly with less intensity than the pulses that were used to cause the visual pigment breakdown) used to illuminate the retinal area of interest, allowing for the measurement of the change in reflection of the area of interest and, thus, the content of the visual pigment. The wavelength of the illuminating light could be the same as the initial bleaching light or the illuminating light could be of different wavelength than the bleaching light. One exemplary pulse sequence comprises one to four strong pulses, to heavily bleach the visual pigment, and then a series of low intensity pulses applied over a selected period of time to allow images to be made. The change in reflected light is measured via these images, and the change versus time indicates the rate of regeneration, as illustrated in FIG. 5. By measuring the slope of the regeneration, the glucose concentration can be calculated. The higher the slope of the regeneration of the visual pigment, the higher the concentration of glucose. This curve is not necessarily linear, and the actual measured reflectance of the retina decreases as regeneration proceeds.

The wavelength of light chosen for the illumination pulses may be any wavelength that would be absorbed by any visual pigment. In a preferred method, narrow band light that is absorbed by either green visual pigment or red visual pigment may be used. It is preferable to avoid light in the blue range, since blue light is mote highly scattered by cataracts than the longer visual wavelengths; cataracts being a common malady in diabetic patients. The device may either use polychromatic light (e.g., the white light that is contained in currently marketed retinal cameras) for the pulse sequence, with the light then being filtered at the CCD or narrow-band light specifically chosen for a particular visual pigment (e.g., 540 nm light for bleaching of the green visual cone pigment) for use as the illumination light. Narrow band light has two advantages. First, narrow band light is generally more comfortable for the patient and, secondly, the pupil does not react with as much constriction to each pulse of narrow band light as compared to broad-band light.

A background blue light may be used throughout the testing period to reduce the effect of the rod visual pigment, by keeping these pigments in a constant bleached state. Since the regeneration rate of this rod pigment is thought to be slower than cone visual pigment, the addition of pigments of differing regeneration times may lessen the accuracy of the measurement without this feature.

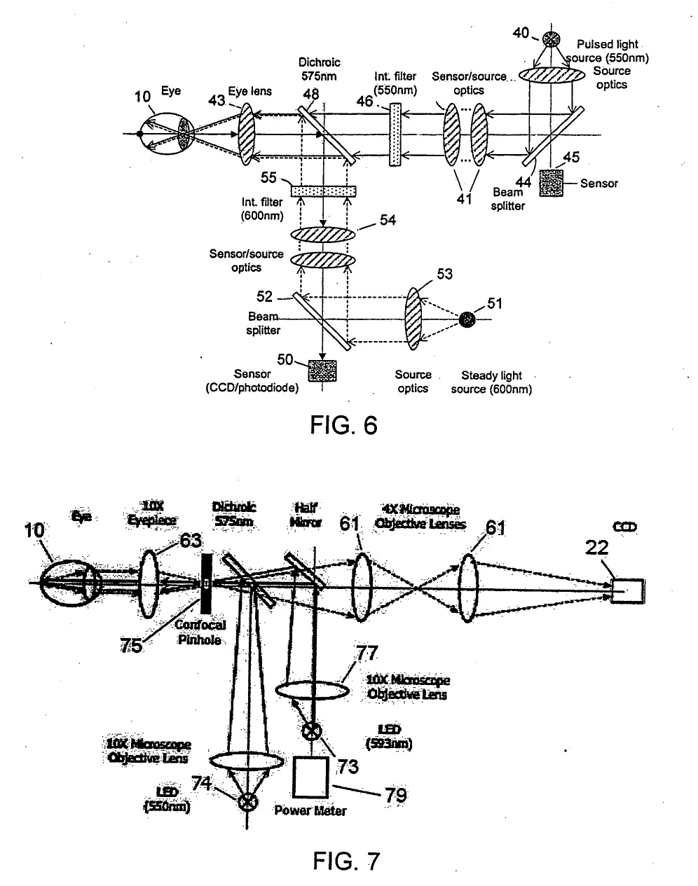

A further embodiment of the optics system 11 and illumination system 12 is shown in FIG. 6. This configuration provides a light source at one wavelength and a sensor system that operates with its own separate light source at a second wavelength. The use of two wavelengths completely separates and isolates the bleach light source from the sensitive measurement process. Thereby, a sensor that does not respond to the bleaching wavelength does not sense the bleaching light and its output can be amplified for the reflected light at a second wavelength.

In the horizontal path with the eye 10, a pulsed light source 40 is imaged into the pupil of the eye with sensor/source optics 41 and an eye lens 43. A sensor 45, near the pulsed source, is used only for feedback control of the pulsed source and receives light through a beam splitter 44. The pulsed source 40 is filtered by an interference filter 46 at 550 nm and the filtered light passes through a dichroic beam splitter 48, and then travels through the eye optics 43 and into the eye 10. This source and path accomplishes bleaching of the visual pigments with high intensity light. The bleached area is then monitored over time by sensor 50 coupled with lower intensity light at the second wavelength. The rate of recovery or rate of regeneration of the visual pigment is the parameter that is used to calculate the glucose level.

With reference to FIG. 6, the light path for measurement of the visual pigment regeneration (light going through elements 54 and 55) is provided to sense the very low reflected light levels without the interference of the bleaching light, which may be of a different wavelength. This can be accomplished by operating a steady light source 51, with source optics 53, to illuminate the back of the eye at a significantly different wavelength to allow for total blocking of the 550 nm pulsed source. The source 51 light is combined with the sensor path with a beam splitter 52 passing through optics 54, and then is filtered to a narrow range preferably around 600 nm by interference filter 55. The source 51 light is focused at the pupil of the eye to provide light to a broad area of the retina. The sensor path may operate at 600 nm with the use of a filter 55, or at a wavelength significantly different than the wavelength of the pulsed source. A wavelength near 600 nm is a preferred choice because the long wavelength pigments in the cones are still very sensitive at 600 nm and the blood vessels in the retina absorb relatively little light. The steady light from the source 51 is at a low level that does little bleaching. The sensor 50 is conjugate with the retina of the eye and is thereby in focus with the retina. The sensor 50 can be, for example, a CCD, CMOS imager, or a photodiode. The photodiode can be a more sensitive device than a standard CCD and it can be utilized in the frequency domain to filter out all of the first order effects and only look at the higher order harmonics as described in the above-referenced U.S. Pat. No. 6,650,915, or to make other time-based, frequency-based, or phase-based measurements.

With reference to FIG. 7, another embodiment of the invention uses a pinhole 75 located confocally with respect to the retinal image. Light is projected into the eye through this pinhole aperture and reflected light from the retina is collected back through it. The confocal pinhole 75 serves to limit the spatial extent of the light on the retina. The size of the pinhole 75 may be changed to suit the requirements. For instance, it may be beneficial to illuminate only the foveal spot on the retina. By avoiding the illumination outside the fovea, bleaching of rods would be minimized. Since cones regenerate faster than rods, this would expedite the measurement process. Alternatively, it might be preferable in some subjects to make the measurement outside the fovea. This could be especially true in subjects with macular degeneration. In this case, the confocal pinhole 75 could be annular in shape, allowing measurement of a spatial ring outside the fovea. Also, the confocal pinhole 75 could contain a multiplicity of segments or holes. This would allow different portions of the retina to be illuminated by different types or levels of light. For instance, two spots of light could be projected onto the retina. The retinal reflectance would change in response to this light, and achieve a steady state after a period of time. Either during this equilibration process, or upon achieving steady state, the reflectance from these two or more spots is measured. The reflectance values and the difference between them are correlative with the level of blood glucose and can be used to measure the blood glucose level. The multiplicity of spots can be projected onto the retina in any arbitrary pattern, possibly as an array of spots in a grid, or as segments of a circular spot. The light spots can be detected either with discrete detectors or with a single array detector such as a CCD array. The measurement method described here can give a very rapid measurement of blood glucose. As equilibration is reached over a short period of time, the noise in the measurement decreases. In addition, this measurement, made in a light adaptation (bleaching) phase, can be made at relatively high light levels compared to measurements made purely in the regeneration, or dark adaptation, phase.

In the embodiment with CCD or CMOS imaging, image analysis tools available in commercially available software packages such as MATLAB can be used. With these tools, the image overlay can be accomplished so that the exact area is repeatedly measured. The initial image capture can be accomplished with a commercially available data capture board (e.g., a National instruments NI 1409 installed in a PC) and the mathematical tools in MATLAB can then be used to analyze the trends in the regeneration rates and to convert those values to glucose levels.

In one variation of the photodiode measurement of the reflectance, a CCD or similar device is used to “steer” the photodiode to the area of interest (e.g., the fovea). The photodiode integrates the signal from an area whereas the CCD provides an image. If the CCD is sensitive enough, it is preferred because the formation of an image allows the definition of an area to be measured, and that area can be repeatedly measured. If a photodiode is used, it may need to be aligned to the spot to be measured, which can be done with known servo methods.

A consideration in making comparable measurements is the variation in light that illuminates the area of interest due to the pupil changing size and to head/eye movement during the capture of the repeated images. This variation can be minimized by also making measurement of a non-changing target in the back of the eye. The optic disk is a good choice of an area to measure and may be used as a reference. For example, this may be done by calculating a ratio of the light returned from the measurement area to the light returned from a defined area of the optic disk. The optic disc is area of the retina where the optic nerve enters the eye. It contains nerve fibers but no cones or rods. Another way to establish a reference is to take measurements at two wavelengths of light, with one wavelength selected for strong absorption by a cone visual pigment, e.g., green at 540 nm, and the second at a non-absorbing point, e.g., 800 nm. The area of the retina to be used for image stabilization can be illuminated by light of a wavelength outside the wavelengths absorbed by visual pigment, and spatially or spectrally distinct from the area used to measure regeneration. For instance, near infrared wavelengths longer than 700 nm can provide excellent contrast of retinal vasculature. An annular ring image using such near infrared wavelengths could be used.

In embodiments that use imaging, bleaching can be done over a greater area than that which is to be measured. By establishing datum points from a first image following bleaching, and then measuring the darkness of a defined area relative to the datum points, subsequent measurements can again measure the same area by reference to the datum points. Alternatively, the first image can be used as a filter which is passed over the subsequent data, and by known image processing methods of translation, rotation, and scaling, the exact overlay can be obtained to thereby locate the same area. The measure of brightness of the defined area is accomplished by summing the value of all of the pixels of the camera in the defined area.

FIG. 7 illustrates an exemplary apparatus to quantitatively measure light reflected from the human retina. The device uses an imaging CCD camera 22, onto which an image of the retina is placed. A region of interest can be selected based on the experimental requirement. For example, the device can image a spot of the retina that is physically 0.6 mm in diameter. A larger spot can be imaged using a larger pinhole aperture. Although FIG. 7 shows a second LED 74 that could be used for measuring regeneration at a second wavelength, in the examples that follow, a single LED 73 with a wavelength of 593 nm was used as illumination for both the bleaching phase and for the regeneration phase.

The head is brought into position and rested in a head restraint consisting of an adjustable chin rest and forehead strap. The head restraint is adjusted to bring the eye to a position where it is possible to look into an eyepiece 63. The eyepiece 63 can be a standard 10× wide field microscope eyepiece, such as the Edmund #A54-426. The retina is illuminated with light from a 593 nm wavelength LED 73, such as a LumiLEDS #LXHLMLIC LED with adjustable intensity controlled from a DC power supply (e.g., CIC PS-1930). The output of the LED 73 can be measured with a power meter 79, such as the Melles Griot 13PDC001. The LED emission is collected with a 10× microscope objective lens 77, such as Edmund #36-132. The LED 73 is re-imaged onto the reticle plane of the eyepiece 63. For example, a 1 mm pinhole aperture 75 is located at this reticle plane, and serves as a confocal aperture. The area of the illumination is limited by this aperture to 1 mm. The magnification power of the eyepiece 63 and of the human eye combine to make the final image diameter on the retina equal to 0.6 mm diameter in this example. The power meter 79 is used to adjust the power density at the retina from LED 73 to the level required for either the bleaching or regeneration phase; in this example 5.8 or 4.2 log Trolands, respectively. (Troland is a unit of measure of retinal illuminance defined as 1 candle/m² on a sufface viewed through an artificial pupil of area A=1 mm².)

The subject is directed to look forward into the eyepiece 63, so that the image of the pinhole is centered in his field of view. As a result, the light is imaged onto the foveal spot of the retina. A portion of the illuminating light is reflected by the retina and passes out through the pupil of the eye, through the eyepiece 63 and is imaged confocally onto the 1 mm pinhole. The light passed by the pinhole then impinges on two 4× microscope objective lenses 61, such as Edmund #36-131 lenses acting as a relay lens system. The image is carried along further and eventually the retina and pinhole are imaged onto the active element of the CCD camera 22, such as a Pulnix #TM-1020 CL or DVC #1412 AM camera.

The digital images are collected from the camera 22 using a CameraLink™ frame grabber, such as National Instruments #1428 installed in a PC. The files are saved as discrete images and formed into a multi-layer file. An exemplary analysis procedure is as follows. The camera 22 is set to the highest gain setting and binning is set to 2×2. A series of raw images is collected. Initially the LED is at low intensity. After 2-3 seconds the LED is switched to high intensity and left high for 20 seconds for the bleaching phase, then switched low again. The regeneration is measured for about 40 seconds at the low light intensity. The data collection results as a series of image files. A 40×40 pixel region of interest (ROI) is defined, in the center of the bleached fovea. The mean intensity within the ROI is found for each image, and the mean intensity data are exported to a spreadsheet program for display and analysis.

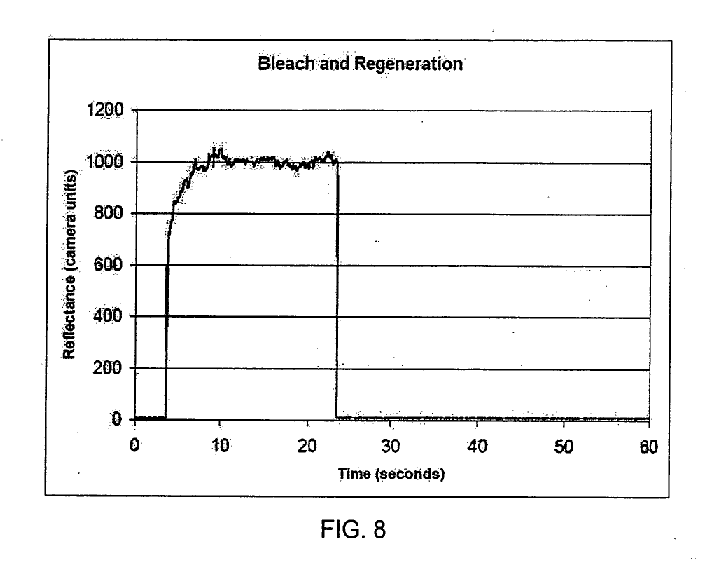

FIG. 8 shows a graph of an example trace. Each data point is the mean intensity within a region of interest in a camera frame. The camera frame rate is 20 frames per second. The x-axis shows time in seconds. The y-axis shows mean pixel intensity in camera units. In FIG. 8, it can be seen that when the LED is switched to the bright setting at about the 3 second point, the measured signal first increases rapidly, but then a slower increase in retinal reflectance (due to bleaching) can be observed. When the LED is switched low at 23 seconds, the regeneration of visual pigment can be followed. Intensity points immediately before and immediately after the light is switched from high to low intensity can be used to photometrically correct the measurement system, since the ratio of the input light intensities is known with a high degree of accuracy. The ratio of the reflected and measured light intensities should have the same ratio, assuming that the measurement circuitry is linear. If the ratio is not the same, it can be due to the introduction of an offset on the intensity axis. An algorithm can be used to remove any offset, thereby creating an intensity axis in true spectroscopic units of percent reflectance, as a percentage of the full bleach. This technique could be considered to achieve the same result as having measured a background trace at full bleach, but it arrives at a photometrically accurate result without degrading the signal-to-noise ratio of the data from division by a second noisy signal.

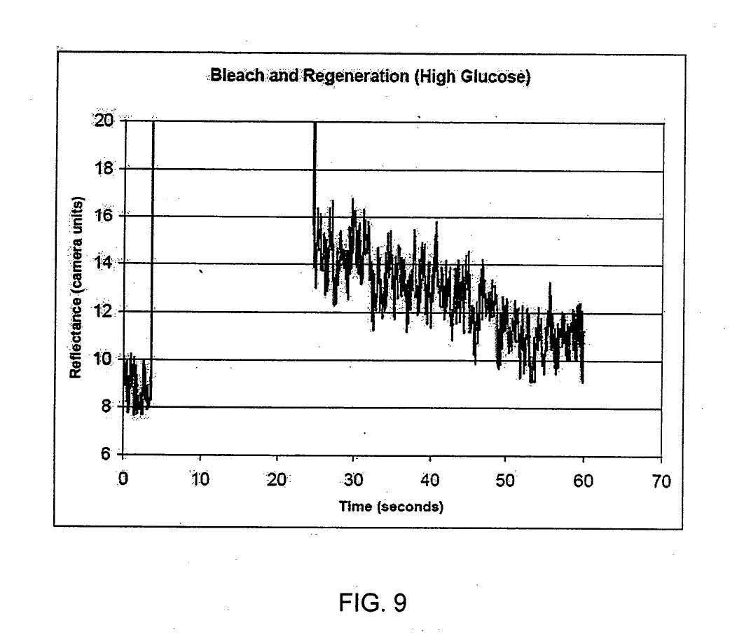

FIG. 9 illustrates an expanded view of a portion of the graph of FIG. 8, showing the lower level reflectance values in greater detail. In the above experiment, the glucose level of the subject was 123 mg/dl. At the start of the experiment, the reflectance of the fovea is relatively low, measuring about 9 camera counts. The subject had been in a normally lit room prior to the experiment. The reflectance level can be considered indicative of the reflectance level of the retina for this subject in normal room light. At the 3 second point, the LED is turned high and the retina begins to be bleached, thus becoming more reflective. When the LED intensity is returned to the original level, it can be seen that the reflectance of the retina is higher than it was before, now measuring about 15 counts. Over time, the reflectance decreases, following a fairly linear slope until 55 seconds, where it proceeds at a slower rate of regeneration.

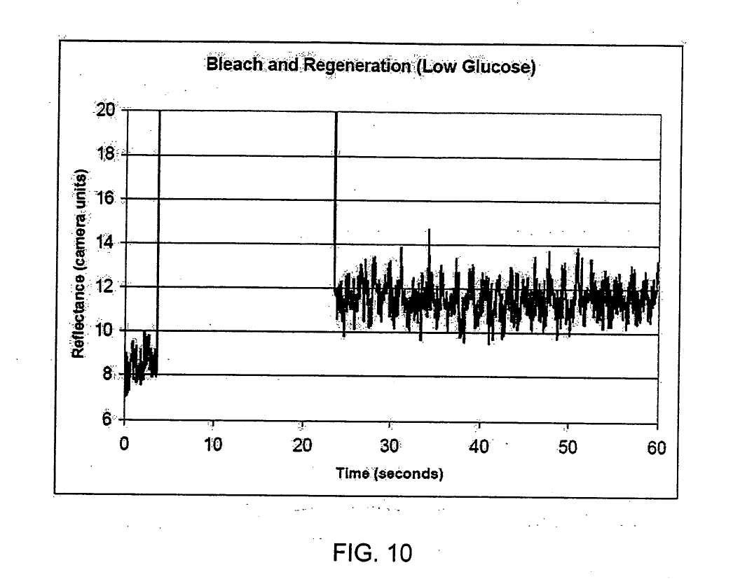

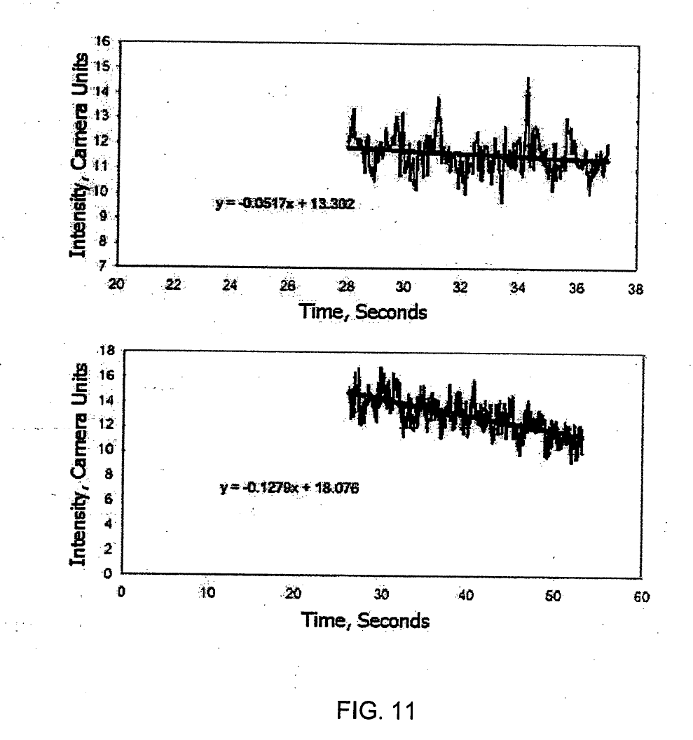

FIG. 10 shows a graph depicting measurement from the same subject, when his glucose level is low, at 81 mg/dl. In this measurement, reflectance again starts out low, at 8-9 camera counts. Following the bleach event, the reflectance is about 11-12 camera counts. Instead of rapidly decreasing, the reflectance remains near this level over the course of the remaining roughly 40 seconds. The initial downward slope of the regeneration curve following bleach is the quantity that is used to correlate with glucose level. A linear portion of the regeneration data near the beginning of the post-bleach phase is extracted and a best-fit line is calculated. For the two traces described with reference to FIGS. 9 and 10, the linear fits are shown in FIG. 11, where the top graph is a low glucose reading (81 mg/dl) and the lower graph is a higher glucose reading (123 mg/dl).

Pulsed Techniques

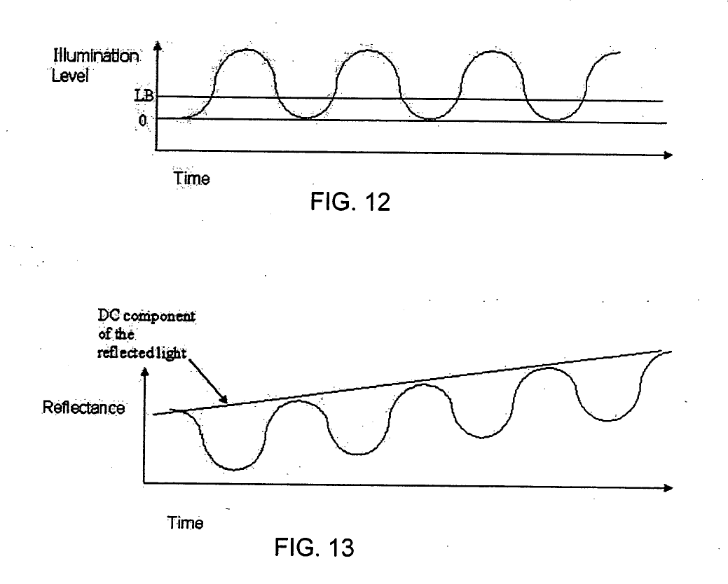

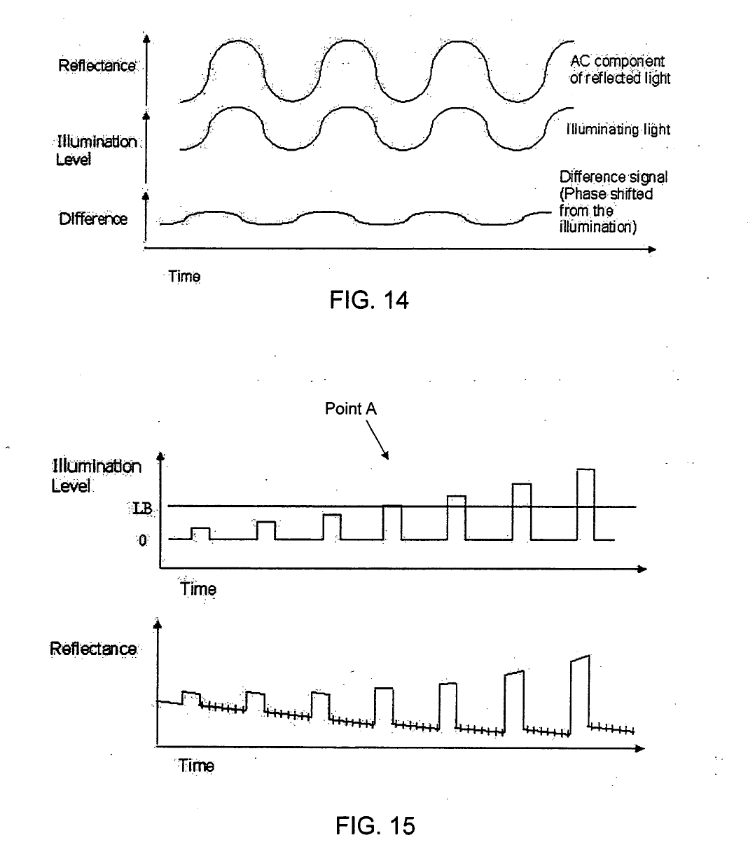

At the start of a testing sequence, the fovea is always at some level of bleaching—neither heavily bleached nor completely dark-adapted. This initial equilibrium level can be referred to as the “level of bleaching” or “LB”. If the eye is illuminated with a time-varying light as illustrated in FIG. 12 with little or no light as the lowest level and the maximum well above LB, there is bleaching whenever the light level is above LB, and regeneration when it is below (the time varying light can be light modulated by a sinusoid, sawtooth, square-wave or other waveforms). However, there is still bleaching when the input signal decreases below the maximum (until it drops below LB), and there is regeneration whenever the light drops below LB. Since regeneration can only proceed at a rate dependent on the glucose level, but bleaching can be much more rapid depending on intensity of the illumination, there would ordinarily be a gradual net increase in reflectance. As time proceeds, depending on both the minimum and maximum magnitude of the time-varying light, the overall reflectance level could increase continuously, yielding a ramp with a variation imposed on it, as illustrated in FIG. 13.

The changes in reflectance also result in a phase shift between the reflected light and the illuminating light, the magnitude of which corresponds to bleaching and regeneration rates, both of which are indicative of the glucose level. In addition, the ramp should also be indicative of the net bleaching rate over time, and this ramp (low frequency or “direct current”) portion of the signal also contains information related to the glucose level. Harmonics or other distortions as disclosed in the above-referenced U.S. Pat. No. 6,650,915, which are part of the high frequency (or “alternating current”) portion of the waveform, are also indicative of the visual pigment bleaching and regeneration rates.

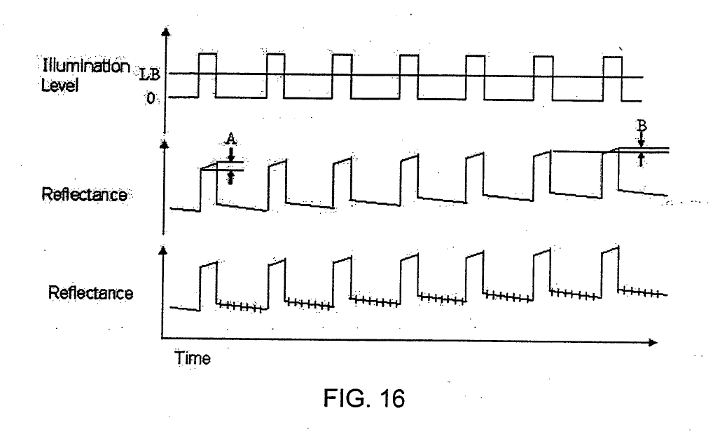

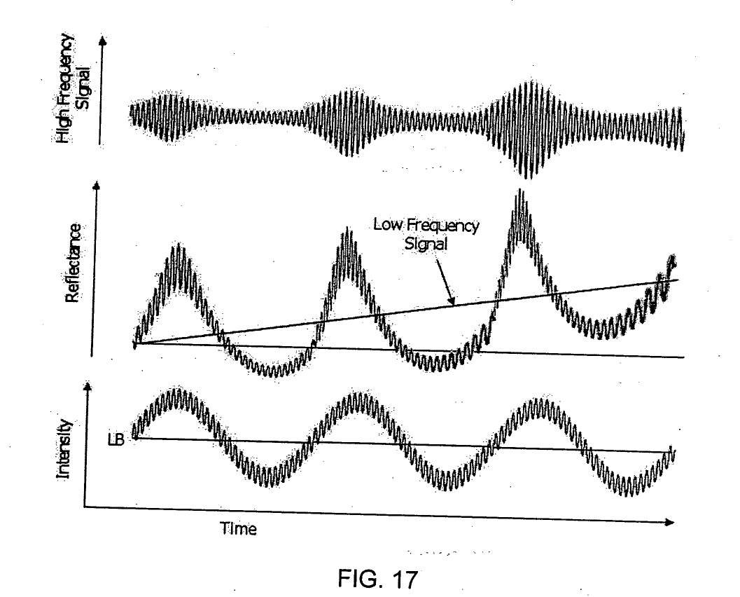

Similarly, if the illuminating light is pulsed, it is possible to make a number of different measurements. One such approach is a series of pulses of increasing amplitude, starting at illumination levels below the LB, and ending at or above it, as shown in FIG. 15. The resulting curve decreases in the time between pulses due to regeneration, and the peaks of the earlier, lower pulses, also decrease at the same rate as when the light is off. When the pulses became large enough that there is net bleaching during the pulse, the amount of reflectance increases during the pulse, but continues to decrease during the off-period. The level of light that corresponds to offsetting the regeneration by bleaching (Point A), the amount of bleaching during the pulses, and the regeneration between pulses (small measuring pulses represented by the “hash marks” in FIG. 15) can all be related to glucose level.

In an alternative embodiment, pulses of a constant level are used, all of which are above the LB, as shown in FIG. 16. Here, the amount (or rate) of bleaching during pulses (difference A), the relative increase in bleaching level from each pulse (difference B), and the decrease between pulses due to regeneration (“hash marks”) can all be related to glucose concentration.

The intensity of the illumination light may also be doubly modulated, at a high frequency and at a lower frequency, as illustrated in FIG. 17. As an example, the high frequency modulation can be 10-20 hertz, and the lower frequency can be 1-2 hertz. If the signal is biased as shown, so that it is above LB for at least part of the low frequency cycle, the bleaching resulting from the part of the cycle above LB would cause a net increase in reflectance during that part of the cycle, as in FIG. 15. The entire signal can be used for determination of glucose, or a known high-pass filter can be employed to isolate the high-frequency portion of the signal. The amplitude of the high-frequency portion of the signal would also increase over time, as the overall reflectance of the retina increased from the net bleaching occurring during each of the low frequency cycles, and the amount of increase would be dependent on glucose concentration. The rate of increase of either the low-frequency portion of the signal or the increase in amplitude of the high frequency portion of the signal could be used to determine glucose concentration.

According to another exemplary embodiment, glucose is measured using the rate of bleaching. Since regeneration is occurring whenever the eye is not completely dark-adapted, faster regeneration reactions which occur at high glucose concentrations would slow the rate of bleaching. This relationship provides a methodology of measuring regeneration rate, and thus glucose. First, the light is brighter and, therefore, easier to see with an inexpensive camera. Second, the reaction goes faster, making the test possibly shorter in duration. Third, there is no need for “registration” of frames between a bleach phase and a regeneration phase. Lastly, regeneration can be measured without causing additional bleaching from the measurement pulses.

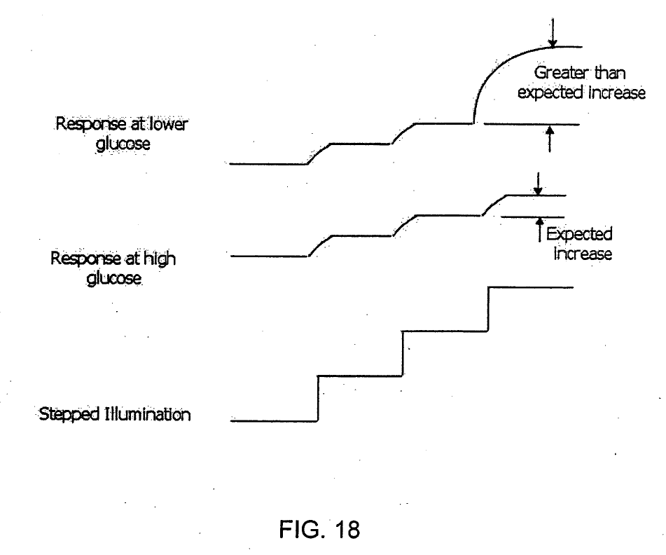

In yet another embodiment, illustrated by FIG. 18, blood glucose can be measured using the regeneration of visual pigments without a “bleaching event.” In one example, referred here to as steady-state regeneration measurement methodology, glucose is measured by determining retinal reflectance at different light levels. This is the equivalent of the color matching methodology described in U.S. patent application 20040087843 A1. At a given light level, if the glucose concentration is high enough to regenerate the pigment at a rate higher than that bleached by the light, a fixed level of reflectance (calibrated for each patient) results. When the light level causes more bleaching than can be regenerated, the visual pigment is depleted faster than it can be made, and the reflectance level rises to a level higher than if a higher concentration of glucose was present. In this method, the retina is illuminated with one light level, a steady state is achieved, and the reflectance is recorded. The retina can be illuminated at a second, increased level, and a new steady state reached. This reflectance is recorded and calculated as a ratio to the first reading. If the light level is still below that which causes more bleaching than regeneration, the expected increase in reflectance results. If, however, the new light level causes more bleaching than regeneration, a higher reflectance than expected would be measured at the new light level. If the light levels are increased in a step-wise fashion, eventually a level is reached where the bleaching effect of the light exceeds the regeneration rate for the patient's glucose level, and a higher than expected increment of reflectance results (a “threshold effect”). Estimation of glucose can be made by considering the light levels below and above the threshold, and from the change in the ratio from the expected amount.

In a second example of measuring blood glucose using visual pigments without a “bleaching event,” a steady-state regeneration measurement methodology uses measurement pulses only to create a steady state of foveal reflectance which corresponded to glucose level. The first pulse increases the reflectance of the fovea, and each pulse is adjusted to maintain the same reflectance. This procedure is repeated at a second illumination level. The levels of reflectance measured during the initial pulse and the second pulse, as well as the ratio of the magnitude of the pulses required to maintain the same reflectance reading at the two levels, are related to glucose concentration.

When glucose measurements are sought, there may be patient-to-patient variability, and the calibration of each device may be required owing to this variability. Also, as the changing state of each patient's diabetes can affect retinal metabolism and thus influence the regeneration rates of the visual pigment, recalibration may be required at periodic intervals. Periodic calibration of the device is useful in patient care as it facilitates the diabetic patient returning to the health-care provider for follow-up of their disease. The device may be equipped with a method of limiting the number of tests, so that follow-up is required to reactivate the device.

In one embodiment of the device, a temperature sensor is employed to sense the body temperature of the individual under test. It may be important to know the body temperature, since temperature may affect the rate of bleaching or regeneration of visual pigments. While any suitable temperature measuring technique could be used, it may be preferable to make a measurement that senses core temperature as closely as possible, and particularly desirable to make an optical measurement. One such method of making an optical temperature measurement uses emission spectroscopy. The optical system already in use for measuring visual pigments could be used to measure energy emitted from the eye with a suitable infrared sensitive photodetector. As predicted from the well-known Planck's quantum theory, the temperature may be measured from the ratio of emitted light at two properly-chosen infrared wavelengths. The measurement process is similar to that found in a commercial ear-cavity thermometer.

In addition to the optical techniques described for measuring the regeneration rate of visual pigments, other technologies may be employed which also are responsive to this rate, and can be used to make measurements that can be related to glucose concentration. One such technique is the “electroretinogram,” as described by O. A. R. Mahroo and T. D. Lamb in a paper entitled “Recovery of the Human Photopic Retinogram After Bleaching Exposures: Estimation of Pigment Regeneration Kinetics, J Physiol., 554.2, pp 417-437. In this technique, the response of the neural system to illumination is indicated by the appearance of an electrical potential at an electrode connected to tissues surrounding the eye, and the level of pigment bleaching or regeneration can be followed by measurement of the electrical activity in response to pulses of dim light after a bleaching event. The rate of regeneration measured by this technique can be related to glucose concentration as described in the optical measurement embodiments.

Similarly, measurements of neural response indicative of visual pigment regeneration can be made using standard techniques for electroencephalography. In this case, electrical measurements of brain waves are made by attaching electrodes to the scalp, and when neural events corresponding to the sensation of light in the retina occur, they can be used to measure the state of bleaching or regeneration of the visual pigments. The rate of regeneration measured by this technique can be related to glucose concentration as described in the optical measurement embodiments.

Owing to the simple optical systems employed in the foregoing embodiments, and the absence of any requirement to separate the different wavelengths of light for spectral analysis, it is practical to make these embodiments from readily-available, lightweight, small optical parts (e.g., a CCD and lenses), and to construct the devices in the form of glasses, goggles sufficiently small and light to be comfortably worn by the user, or in the form of small hand-held devices such as monoculars or binoculars. Similarly, a small head-mounted device with a weight low enough to be comfortably worn by the user can be constructed from these components.

Any of the above-described embodiments which are suitable to measure the regeneration rate of visual pigments can be used to make measurements which are indicative of disease states or conditions of health of the person being measured. One such condition is retinitis pigmentosa, an inherited condition in which a person's vision and visual field gradually deteriorate, due to a loss of functional photoreceptors in the retina. Sandberg et al. have shown in a publication entitled “Acuity Recovery and Cone Pigment Regeneration after a Bleach in Patients with Retinitis Pigmentosa and Rhodopsin Mutations, ” (Investigative Ophthalmology and Visual Science. 1999;40:2457-2461.), that the rate of regeneration for patients with this condition is substantially lower than that of normal patients. Thus, measurements of the rate of regeneration, alone or coupled with measurement of blood glucose by an independent method, can serve as techniques for diagnosing this or other conditions which reflect deviations from the normal functioning of the process of regeneration of visual pigments in the retina.

### Examples of Clinically-Acceptable Glucose Measurements

Table 1 shows the slope (regeneration rate) obtained for 16 regeneration experiments on 6 different days, using three different subjects, with the apparatus depicted in FIG. 7. For these measurements, a single LED with a wavelength of 593 nm and two brightness levels was used for both the initial (bleaching) illuminating phase, at high brightness, and for measurement of reflectance during the subsequent regeneration phase, at low brightness. The bleaching was carried out over a 20-second period, and the slope of each regeneration was subsequently recorded using the CCD array over a period of time, as described above in the detailed description of FIGS. 7 through 11.

TABLE 1

| | | | | | Calculated | Reference |
|---|---|---|---|---|---|---|
| Subject | date | trial# | Slope (cts/sec) | abs slope (cts/min) | Glucose | Glucose |
| RGM | 2-Apr | 1 | −0.1233 | 7.3980 | 129 | 148 |
| | | 2 | −0.0877 | 5.2620 | 113 | 106 |
| | | 3 | −0.0386 | 2.3160 | 89 | 93 |
| | 3-Apr | 1 | −0.1058 | 6.3480 | 121 | 132 |
| | | 2 | −0.0390 | 2.3400 | 90 | 100 |
| | 4-Apr | 1 | −0.0857 | 5.1420 | 112 | 118 |
| | | 2 | −0.0309 | 1.8540 | 86 | 101 |
| | | 3 | −0.0353 | 2.1180 | 88 | 89 |
| RHS | 6-Apr | 1 | −0.0693 | 4.1580 | 104 | 96 |
| | | 2 | −0.331 | 19.8600 | 228 | 163 |
| | | 3 | −0.0391 | 2.3460 | 90 | 109 |
| JW | 8-Apr | 1 | −0.1976 | 11.8560 | 165 | 191 |
| | | 3 | −0.273 | 16.3800 | 200 | 202 |
| RGM | 12-Apr | 2 | −0.0517 | 3.1020 | 96 | 81 |
| | | 3 | −0.0930 | 5.5800 | 115 | 104 |
| | | 4 | −0.1279 | 7.6740 | 132 | 123 |

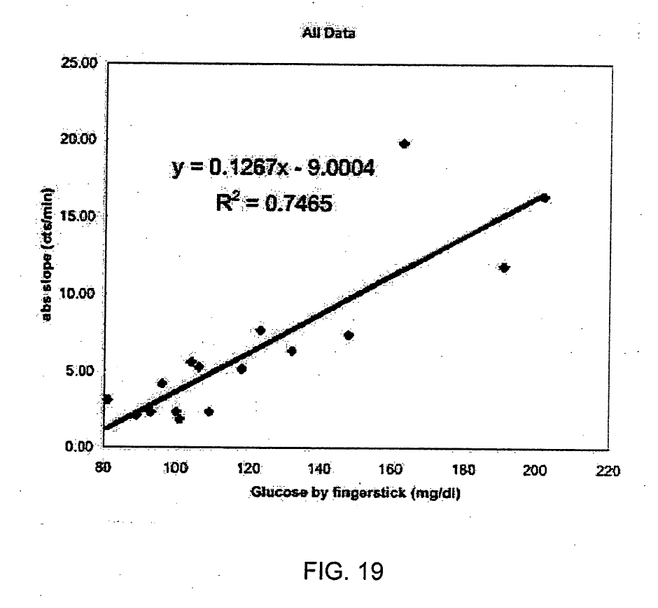

These slopes (or rates) are plotted against the reference glucose concentration, and a best-fit line is computed. These results are shown in a graph depicted in FIG. 19.

The linear fit line is now used to compute a glucose value (x) for a given slope (y). Each of the sixteen experiments is analyzed in this manner, resulting in the “Calculated Glucose” column of Table 1 which may be compared to the “References Glucose” column to the right, which are values obtained for the subjects with a conventional blood glucose meter.

All of these data are plotted on a Clarke Error Grid, shown in FIG. 13. In this graphical grid system, which is used to evaluate the clinical impact of errors in blood glucose measurement, fifteen of the sixteen data points fall in region A, and one data point falls in region B. The regions of the Clarke Error Grid are defined as: A: “Clinically Accurate ” B. “Benign Errors, Clinically Acceptable,” C. “OverCorrection,” D. “Dangerous Failure to Detect and Treat, ” and E. “Erroneous Treatment, Serious Error.” These results therefore constitute clinically-acceptable accuracy for the measurement of blood glucose using the technique.

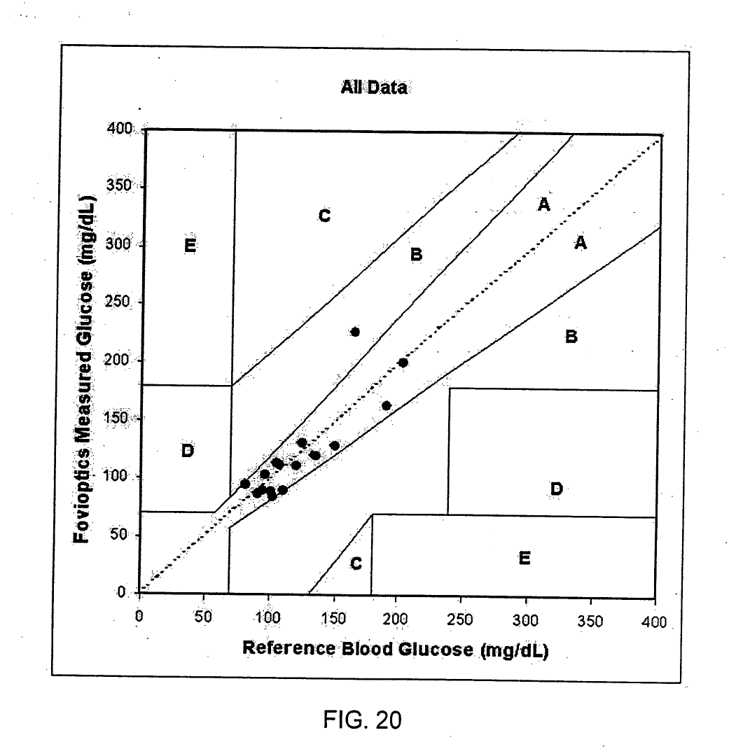

In addition, the data shown in FIG. 20 were collected over the eleven-day period from April 2 through April 12. All the data are plotted on the graph based solely on the reflectance change measured during a period of time, with no intervening calibration or recalibration of the relationship between the rate of regeneration and the corresponding glucose value. Thus, it can be seen that at least over an eleven-day period, there was no need to adjust the response of the measurement due to environmental or physiological changes in the patient, and a recalibration interval for the device or longer than eleven days can be inferred from the accuracy of the results obtained.

It is understood that the invention is not limited to the embodiments described herein to illustrate the invention, aces all forms thereof that come within the scope of the following claims.

1. A method for use in the determination of blood glucose concentration of an individual comprising:
projecting a first light into retina of an eye of the individual;
directing a second light into the retina;
detecting light reflected from the retina from the second light;
measuring a visual pigment regeneration rate in the retina using the light reflected from the retina; and
determining the blood glucose concentration using the visual pigment regeneration rate.

2. The method of claim 1, wherein the second light has a second intensity level, and wherein the second intensity level is lower than the first intensity level.

3. The method of claim 1, wherein the second light is directed into the retina over a selected period of time such that visual pigment is not significantly bleached.

4. The method of claim 1, wherein the first light contains light of a visual wavelength.

5. The method of claim 1, wherein the second light projected into the retina is in the form of pulses of light.

6. The method of claim 1, wherein the second light projected into the retina is in the form of steady-state light.

7. The method of claim 1, wherein the second light projected into the retina is in the form of a time-varying or modulated light.

8. The method of claim 1, wherein the second light has a variable intensity level.

9. The method of claim 1 wherein the first light has a first intensity level selected to bleach visual pigment in the retina of the eye.

10. The method of claim 1, wherein the step of measuring the visual pigment regeneration rate is performed with a photodetector.

11. The method of claim 1 further comprising forming an image of at least a selected area of the retina.

12. The method of claim 1, wherein the first light and second light are of different wavelengths.

13. The method of claim 1, further comprising maintaining a consistent area of measurement in the retina of the eye using retinal feature identification.

14. The method of claim 1, wherein measuring visual pigment regeneration rate comprises measuring light reflected from foveal region of the retina.

15. The method of claim 14, further comprising obtaining images of the foveal region using a photodetector array.

16. The method of claim 14, wherein measuring the light reflected from the foveal region is performed utilizing a photodiode.

17. The method of claim 16, wherein measuring the light reflected from the foveal region comprises measuring light reflected from a central area of the foveal region to account for movement of the retina and the foveal region.

18. The method of claim 1, wherein measuring the visual pigment regeneration rate is performed using cone receptors regeneration rate.

19. The method of claim 18, further comprising illuminating the retina with blue light while measuring the visual pigmentation regeneration rate.

20. The method of claim 1, further comprising using a near-infrared light source for observing retinal features while measuring the visual pigment regeneration rate.

21. The method of claim 20, wherein the near-infrared light source is directed to a region of the retina spatially distinct from the foveal region.

22. The method of claim 20, wherein the near-infrared light source uses a wavelength that does not bleach visual pigment.

23. The method of claim 1 further comprising measuring a temperature of the individual during the measurement of the visual pigmentation regeneration rate.

24. The method of claim 23, wherein measuring the temperature comprises optically measuring the eye.

25. The method of claim 23, wherein measuring the temperature of the individual is used to correct variations in the visual pigment regeneration rate.

26. The method of claim 1, further comprising projecting light through a pinhole aperture.

27. The method of claim 1, further comprising projecting light through a confocal aperture to foveal region of the retina.

28. The method of claim 27, wherein the light has a circular extent, and wherein the light comprises a single spot.

29. The method of claim 27, wherein the light comprises a plurality of spots on the retina, and wherein each spot of the plurality of spots has a different intensity level.

30. The method of claim 29, wherein the plurality of spots are deployed in a radially symmetrical pattern.

31. The method of claim 29, wherein the plurality of spots are deployed in a grid pattern.

32. The method of claim 1, wherein visual pigment regeneration rate is measured by a rate at which the light reflected from the retina decreases.

33. The method of claim 1 further comprising correcting a refractive error in the retina of the individual.

34. An apparatus for determining blood glucose concentration in an individual, the apparatus comprising:
a first light adapted to project a first light into retina of an eye of the individual;
a second light adapted to project a second light into the retina of the eye;
a light detector adapted to detect light reflected from the retina from projecting the second light; and
a processor with programmed instructions adapted to calculate the blood glucose concentration using the light reflected from the retina.

35. The apparatus of claim 34, wherein the first light has a wavelength that is absorbed by visual pigment in the retina of the eye.

36. The apparatus of claim 35, wherein the first light has an intensity level sufficient to bleach the visual pigment in the retina.

37. The apparatus of claim 34, wherein the second light has a wavelength that is absorbed by visual pigment in the retina of the eye.

38. The apparatus of claim 34, wherein the processor analyzes the light reflected from the retina to determine visual pigment regeneration rate in the retina.

39. The apparatus of claim 38, wherein the processor measures the visual pigment regeneration rate using a rate at which the light reflected from the retina decreases.

40. The apparatus of claim 38, wherein the processor calculates the blood glucose concentration using the visual pigment regeneration rate.

41. The apparatus of claim 34 further comprising a pinhole aperture through which the second light passes.

42. The apparatus of claim 34 further comprising a confocal aperture to focus the second light to foveal region of the retina.

43. The apparatus of claim 34, wherein the second light comprises a single light pulse.

44. The apparatus of claim 34, wherein the second light comprises a plurality of light pulses on the retina.

45. The apparatus of claim 44, wherein each light pulse of the plurality of pulses has a different intensity level.

46. The apparatus of claim 34 further comprising means for correction of refractive error in the eye of the individual.

47. The apparatus of claim 34, wherein the first light and the second light have different wavelengths.

48. The apparatus of claim 34, wherein the first light, the second light, the light detector, and the processor form an integrated unit configured to be worn by the individual.

49. The apparatus of claim 48, wherein the integrated unit comprises a form of glasses or goggles.

50. The apparatus of claim 48, wherein the integrated unit weighs less than ten ounces.

51. The apparatus of claim 48, wherein the integrated unit weighs less than sixteen ounces.

52. The apparatus of claim 48, wherein the integrated unit occupies a volume of less than twelve cubic inches.

53. The apparatus of claim 48, wherein the integrated unit occupies a volume of less than forty cubic inches.

54. The apparatus of claim 48, wherein the integrated unit has a form of a hand-held monocular device.

55. The apparatus of claim 48, wherein the integrated unit has a form of a hand-held binocular device.

56. The apparatus of claim 48, wherein the integrated unit comprises a form of a head-mounted apparatus.

57. A device for optical measurement of blood glucose concentration in an individual in a noninvasive manner, the device comprising:
a light projector adapted to project light into retina of the eye of an individual, the light having a wavelength that is absorbed by visual pigment in the retina of the eye;
a light detector adapted to detect light reflected from the retina of the eye; and
a processor with programmed instructions adapted to calculate the blood glucose concentration utilizing a measurement of the rate of regeneration of visual pigment in the retina.

58. The device of claim 57, wherein the light projector, the light detector, and the processor form a head mounted device.

59. The device of claim 57, wherein the light projector, the light detector, and the processor are contained within an integrated unit.

60. The device of claim 59, wherein the integrated unit weighs less than ten ounces.

61. The device of claim 59, wherein the integrated unit weighs less than sixteen ounces.

62. The device of claim 59, wherein the integrated unit occupies a volume of less is than twelve cubic inches.

63. The device of claim 59, wherein the integrated unit occupies a volume of less than forty cubic inches.

64. The device of claim 59, wherein the integrated unit has the form of a pair of glasses or goggles.

65. The device of claim 59, wherein the integrated unit having the form of a hand-held monocular device.

66. The device of claim 59, wherein the integrated unit having the form of a hand-held binocular device.

67. The device of claim 57, wherein the light projector projects steady state light.

68. The device of claim 67, wherein the processor determines the rate of regeneration of the visual pigment by measuring retinal reflectance.

69. The device of claim 57, wherein the processor determines the rate of regeneration of the visual pigments using an electroretinogram.

70. The device of claim 57, wherein the processor measures the rate of regeneration of the visual pigments using an electroencephalogram.

71. The device of claim 57, wherein the light has a visual wavelength.
